(19) United States
(12) Patent Application Publication
(10) Pub. No.: US 2025/0127499 A1
(43) Pub. Date: Apr. 24, 2025

(54) ENDOLUMINAL TREATMENT DEVICES AND RELATED METHODS

(71) Applicant: Boston Scientific Scimed, Inc., Maple Grove, MN (US)

(72) Inventor: Martyn G. FOLAN, Co. Galway (IE)

(73) Assignee: Boston Scientific Scimed, Inc., Maple Grove, MN (US)

(21) Appl. No.: 18/923,968

(22) Filed: Oct. 23, 2024

Related U.S. Application Data
(60) Provisional application No. 63/592,758, filed on Oct. 24, 2023.

(51) Int. Cl. A61B 17/00

(52) U.S. Cl. CPC A61B 17/0057; A61B 2017/00597; A61B 2017/00623; A61B 2017/00632; A61B 2017/00659; A61B 2017/00676

(57) ABSTRACT

Medical devices and related methods are described including a medical device including a tube defining a lumen and configured to couple to a source of suction, and a porous body coupled to a distal end of the tube. The porous body may include openings in fluid communication with the lumen. Upon application of a tension force to the porous body, at least a portion of the porous body may be configured to expand outward in a direction that is at least partially perpendicular to a direction of the tension force.

### CROSS-REFERENCE TO RELATED APPLICATIONS

This application claims the benefit of priority to U.S. Provisional Application No. 63/592,758, filed on Oct. 24, 2023, the entirety of which is incorporated herein by reference.

### TECHNICAL FIELD

Various aspects of this disclosure relate generally to minimally invasive medical devices and methods. In particular, aspects of the disclosure relate to medical devices and methods for endoscopic medical procedures, such as closing a wound or otherwise treating tissue.

### BACKGROUND

Endoscopic and open surgical procedures of the gastrointestinal (GI) tract include, for example, colonic resection, bariatric surgery, esophagectomy, gastric bypass, and sleeve gastrectomy, among others. These procedures may result in perforation, post-surgical anastomotic leaks, or other wounds of the GI tract. Patients with perforations, post-surgical anastomotic leaks, and/or other wounds in the GI tract have high mortality rates with limited treatment options. Options include endoscopic placement of clips or stents, endoscopic sutures or sealants, or surgical re-operation. Surgery is relatively invasive and has high morbidity and mortality rates. While endoscopic stent placement is a less invasive option, the stent can migrate from the intended location and/or wall off infection at a treatment site, inhibiting drainage.

The medical devices and methods of the current disclosure may rectify some of the deficiencies described above or address other aspects of the art.

### SUMMARY

Each of the aspects disclosed herein may include one or more aspects of the features described in connection with any of the other disclosed aspects.

According to some aspects of the present disclosure, a medical device may include a tube defining a lumen and configured to couple to a source of suction. The medical device may include a porous body coupled to a distal end of the tube. The porous body may include openings in fluid communication with the lumen, and wherein, upon application of a tension force to the porous body, at least a portion of the porous body may be configured to expand outward in a direction that is at least partially perpendicular to a direction of the tension force.

According to some aspects, the porous body may include an auxetic material. In some examples, the openings of the porous body may have a re-entrant structure. In some examples, the tension force may be at least one of a proximal force on a proximal end of the porous body or a distal force on a distal end of the porous body. In some examples, the distal end of the tube may be configured to transition from a non-linear shape to a linear shape in order to apply the tension force on the porous body. In some examples, the openings of the porous body may be configured to expand in a direction perpendicular to the tension force upon application of the tension force to the porous body.

In some examples, upon application of a compressive force, the at least the portion of the porous body may be configured to contract inwardly in a direction that is at least partially perpendicular to the compressive force. In some examples, the compressive force is in an opposite direction from the direction of the tension force. In some examples, the medical device may include a suture thread extending proximally from a distal end of the porous body, through the lumen of the tube, and to a proximal end of the tube. In some examples, a distal end of the suture thread is fixedly coupled to the distal end of the porous body. In some examples, upon movement of the suture thread in a proximal direction, the at least the portion of the porous body may be configured to contract inwardly in a direction that is at least partially perpendicular to the proximal direction. In some examples, the porous body may include a first section and a second section. The first section may include an auxetic material and the second section may include a non-auxetic material. The first section may include the at least the portion of the porous body that is configured to expand outward in the direction that is at least partially perpendicular to the direction of the tension force. In some examples, the porous body may further include a third section. The second section may be positioned between the first section and the third section and the third section may include an auxetic material. In some examples, upon application of the tension force, the third section may be configured to expand outward in the direction that is at least partially perpendicular to the direction of the tension force, and the second section may be configured to contract inwardly in a direction that is at least partially perpendicular to the direction of the tension force. In some examples, after application of the tension force, the third section and the first section may have a larger diameter than the second section. In some examples, prior to application of the tension force, the first section, the second section, and the third section may have approximately the same diameter.

According to some aspects of the present disclosure, a medical device may include a tube defining a lumen and configured to couple to a source of suction. The medical device may include a porous body including one or more materials and coupled to a distal end of the tube. The porous body may include openings in fluid communication with the lumen. The porous body may include an auxetic material. In some examples, upon application of a compressive force to the porous body, at least a portion of the porous body including the auxetic material may be configured to contract radially inwardly in a direction that is at least partially perpendicular to the compressive force.

According to some aspects of the present disclosure, a method of treating a target site of a body lumen of a patient via a medical device may include advancing a porous body to the target site. The porous body may be coupled to a distal end of tube. The method may further include supplying a negative pressure to the porous body through a lumen of the tube and pulling the tube in a proximal direction such that at least a portion of the porous body may expand outwardly in a direction that is at least partially perpendicular to the proximal direction. In some examples, pulling the tube in the proximal direction may transition the tube from a non-linear to a linear shape. In some examples, the direction may be a first direction. The method may further include pulling a suture coupled to a distal end of the porous body in the proximal direction, which may cause the at least the porous body to contract inwardly in a second direction that is at least partially perpendicular to the proximal direction.

### BRIEF DESCRIPTION OF THE DRAWINGS

The accompanying drawings, which are incorporated in and constitute a part of this specification, illustrate various aspects of this disclosure and together with the description, serve to explain the principles of the disclosure.

FIG. 1A-1E depict aspects of an exemplary medical device, in accordance with some aspects of the present disclosure.

FIGS. 2A-2C depict an exemplary method of using the exemplary medical device of FIGS. 1A-1E, in accordance with some aspects of the present disclosure.

FIGS. 3A and 3B depict another exemplary medical device, in accordance with some aspects of the present disclosure.

### DETAILED DESCRIPTION

Particular aspects of the present disclosure are described in greater detail below. The terms and definitions provided herein control, if in conflict with terms and/or definitions incorporated by reference.

The terms “proximal” and “distal” are used herein to refer to the relative positions of the components of exemplary medical devices. As used herein, “proximal” refers to a position relatively closer to the exterior of the body or closer to an operator using the medical device. In contrast, “distal” refers to a position relatively further away from the operator using the medical device, or closer to the interior of the body.

As used herein, the terms “comprises,” “comprising,” “including,” “includes,” “having,” “has” or any other variation thereof, are intended to cover a non-exclusive inclusion, such that a process, method, article, or apparatus that comprises a list of elements does not include only those elements, but may include other elements not expressly listed or inherent to such process, method, article, or apparatus. The term “exemplary” is used in the sense of “example,” rather than “ideal.”

Further, relative terms such as, for example, “about,” “substantially,” “approximately,” etc., are used to indicate a possible variation of +10% in a stated numeric value or range.

Endoluminal vacuum therapy (EVT or EVAC, and referred to herein as EVAC) is a procedure to treat wounds, such as post-surgical leaks or perforations in the gastrointestinal tract (GI) following a surgical or endoscopic procedure, such as colonic resection, bariatric surgery, or esophagectomy. In EVAC, negative pressure is delivered to the wound site in the GI tract, for example, through a nasogastric tube having a sponge or sponge-like material or foam (e.g., vacuum sealed foam) sutured at its distal end. A proximal end of the tube may be connected to a collection container. The foam is placed endoscopically into the perforation, leak, or other wound. In some examples, EVAC includes endoluminal placement of a foam or other like material into the wound (e.g., target) site or wound cavity, including a perforation, a leak, a cyst, an anastomosis, etc. Placement of the material may be via a catheter, scope (endoscope, bronchoscope, colonoscope, duodenoscope, gastroscope, etc.), tube, or sheath, inserted into the GI tract via a natural orifice. The orifice can be, for example, the nose, mouth, or anus, and the placement can be in any portion of the GI tract, including the esophagus, stomach, duodenum, large intestine, or small intestine. Placement of the material can also be in other organs reachable via the GI tract (e.g., the colon).

Rat-tooth forceps or another accessory device may be extended through a working channel of the scope and used to guide the foam to the wound site as the scope is navigated to the wound site. Negative pressure then is applied. The foam in the wound, along with the negative pressure, may accelerate healing by encouraging local tissue granulation at the wound site. The foam may be replaced with increasing smaller sizes of foam as the wound heals and closes. Present devices suited for EVAC are limited. For example, friction between a foam and a wall of a working channel may increase the amount of force required to extend the foam through the working channel and to a wound site. Furthermore, a size of a foam used during EVAC may not be suitable for a size of a wound site. For example, a size of a foam smaller than a size of a wound site may result in under-packing of the wound site and an inefficient treatment cycle. Additionally, a size of a foam larger than a size of a wound site may result in over-packing of the wound site and damage to the wound site and/or surrounding vessel walls.

Features of the medical devices and methods herein may improve delivery of a foam to a target site and/or provide a foam with varying properties and/or characteristics suitable for treatment of wound sites or wound cavities of varying sizes. Additionally, components of the medical devices described herein may be packaged as a kit. According to some aspects of the present disclosure, the medical devices may include a porous body (e.g., a foam or a sponge) coupled to a distal end of a tube. The porous body may include one or more auxetic materials and/or structures. Auxetic materials and/or structures may have a range of properties and/or characteristics, including among others, expanding (i.e., becomes thicker) in a direction perpendicular to an applied tension force (e.g., becomes thicker in a lateral or radial direction when stretched in a longitudinal direction), contracting (i.e., becomes thinner) in a direction perpendicular to an applied compressive or inward force (e.g., becomes thinner in a lateral or radial direction when compressed in a longitudinal direction), high energy absorption, and high fracture toughness. With such properties, the porous body may be extended through a lumen of a scope device or other medical device with less resistance and/or expanded at a wound site, for example, in the presence of a negative pressure, improving drainage of the wound site.

The one or more auxetic materials and/or structures may include re-entrant structures (e.g., honeycomb, hexagonal, triangular, star, 3-dimensional), rotating rigid structures (e.g., square, rectangle, rhombi, triangle, tetrahedral), anti-chiral structures, and/or chiral structures (e.g., tessellations). One or more initial materials (e.g., polyurethane, polyester urethane, polyether urethane, silicone rubber, copper) used during a manufacturing process of the porous body may be converted to an auxetic material (i.e., having auxetic structures and patterns) or otherwise modified to have auxetic properties using any suitable technique, such as, for example, fused deposition, thermomechanical conversion processes, chemomechanical means, and/or 3D printing.

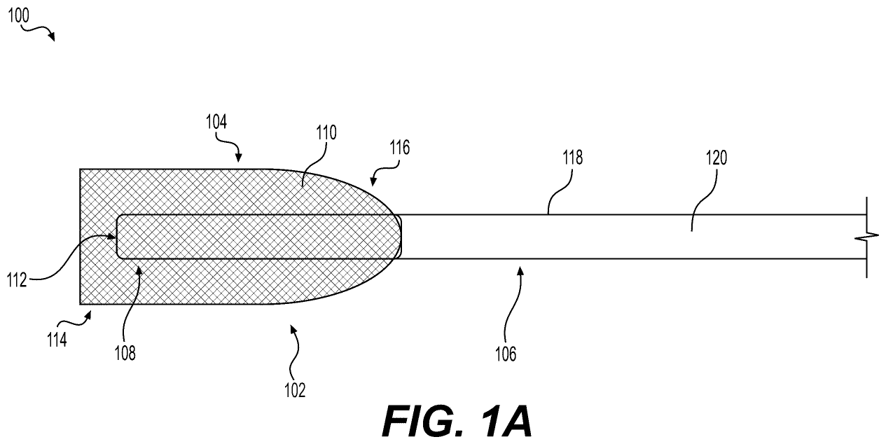

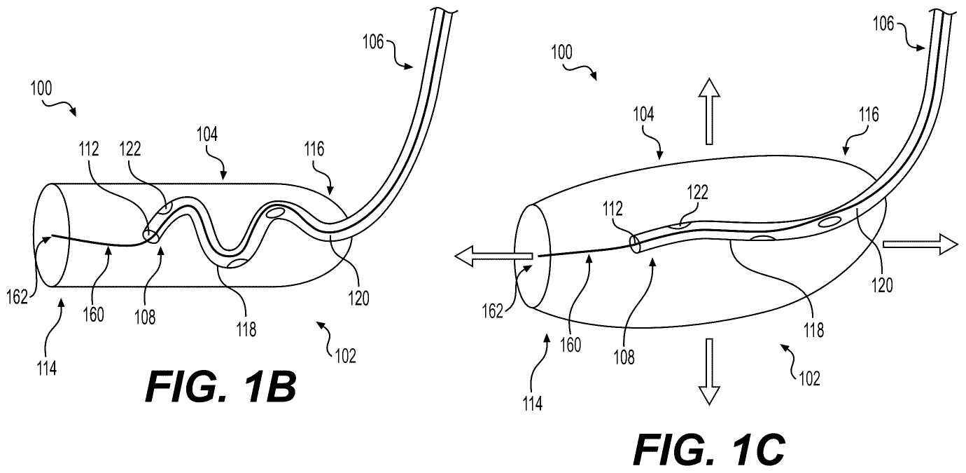

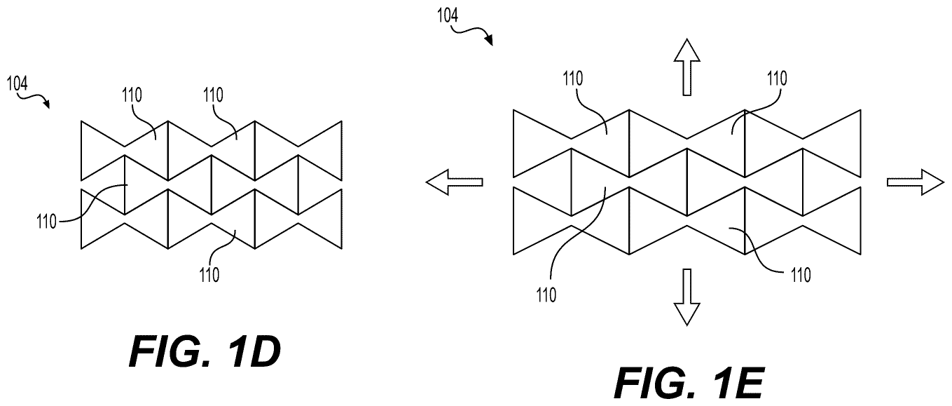

FIGS. 1A-1C illustrate a distal portion 102 of an exemplary medical device 100 that may be inserted into a patient to help the healing of a wound, an anastomosis, or the like, or to otherwise treat tissue. FIGS. 1D and 1E illustrate exemplary features of medical device 100. Medical device 100 includes a porous body (e.g., a foam or a sponge) 104 (hereinafter called “foam 104”) and a tube 106 or other type of conduit defining a lumen. Foam 104 may include one or more auxetic materials and/or structures, having any of the properties discussed above. For example, openings or pores 110 of foam 104 may form a re-entrant honeycomb structure, shown in FIGS. 1D and 1E. Pores 110 may have any suitable size. For example, a size of pores 110 may be selected based on a location of treatment within the body, properties of a wound to be treated, a stage of treatment, or other factors. It will be appreciated that foam 104 may be any shape, including spherical, prismatic, cylindrical, cuboidal, irregular or the like.

Foam 104 may be configured to transition between a relaxed, non-activated configuration (shown in FIGS. 1A and 1B) and an expanded, activated configuration (shown in FIG. 1C). Tension may be applied to foam 104 in one or more longitudinal directions to transition foam 104 from the relaxed, non-activated configuration to the expanded, activated configuration. For example, foam 104 may have an at least partially proximal force exerted on a proximal end 116 of foam 104 and/or an at least partially distal force exerted on a distal end 114 of foam 104 indicated by the left and right arrows in FIG. 1C) to expand foam 104 in one or more lateral or radial directions (perpendicular to a longitudinal axis of foam 104, as indicated by the up and down arrows in FIG. 1C) away from the longitudinal axis of foam 104. In other words, foam 104 may expand in a direction perpendicular to a direction of a tension force applied or at least a portion of foam 104 may expand in a direction at least partially perpendicular to a direction of a tension force applied. A size of pores 110 in the expanded, activated configuration (shown in FIG. 1E) may be larger than a size of pores 110 in the relaxed, non-activated configuration (shown in FIG. 1E).

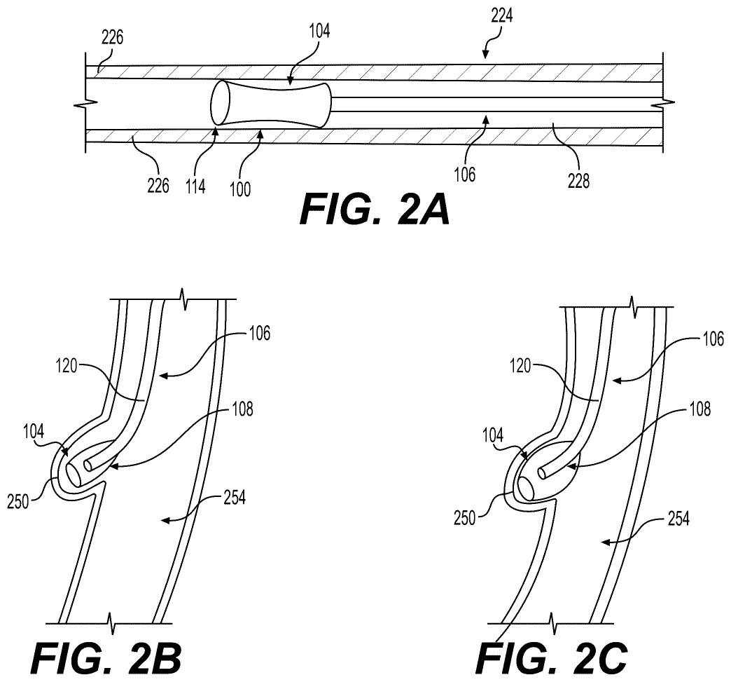

Additionally, foam 104 may be configured to transition between the relaxed, non-activated configuration to a contracted configuration (shown in FIG. 2A). A compressive or inward force may be applied to foam 104 in one or more longitudinal directions, for example, to compress and/or contract foam 104 in one or more lateral or radial directions (i.e., cause foam 104 to become thinner) towards the longitudinal axis of foam 104. A direction of a compressive or inward force applied to foam 104 may be opposite of a direction of a tension force applied to foam 104. For example, a distal force may be applied to proximal end 116 of foam 104 and/or a proximal force may be applied to distal end 114 of foam 104. Foam 104 may compress and/or contract in a direction perpendicular to a direction of a compressive force applied or at least a portion of foam 104 may compress and/or contract in a direction at least partially perpendicular to a direction of a compressive force applied.

It will be appreciated that the one or more forces described above (e.g., compression, tension, etc.) may be selectively applied to foam 104 (e.g., a user pulling a thread or a tube in a proximal direction relative to foam 104, which will be discussed in greater detail below, a user turning on a vacuum or suction source) or non-selectively applied to foam 104 (e.g., a force applied to foam 104 by a wall defining a lumen of a scope device or other medical device, a force applied to foam 104 by a wall of a body lumen) at the same or different times providing varying sizes of foam 104 during an EVAC procedure.

Foam 104 may be fixedly or releasably attached to a distal end 108 of tube 106. For example, distal end 108 of tube 106 may be attached to foam 104 via sutures or other ties, an adhesive, a shrink-wrapped material, elastic(s), or the like. In one example, a recess (not shown) may be provided in foam 104 (e.g., proximal end 116 of foam 104) to receive distal end 108 of tube 106. Tube 106 may include an outer wall 118 defining a lumen 120. Lumen 120 may be open at both a proximal end (not shown) and distal end 108 of tube 106. Although tube 106 is illustrated as terminating proximal to distal end 114 of foam 104 in FIGS. 1A-1C, it will be appreciated that tube 106 may extend fully through foam 104 such that a distal opening 112 of tube 106 is aligned with or adjacent to distal end 114 of foam 104. Tube 106 may be sufficiently flexible so that distal end 108 of tube 106 may transition between a sinuous, kinked, wavy, curved, or other non-straight shape (as shown in FIG. 1B) and a substantially straight or linear shape (as shown in FIG. 1C). Straightening or stretching distal end 108 of tube 106 by, for example, pulling tube 106 proximally, may also apply tension to foam 104 in one or more longitudinal directions transitioning foam 104 from the relaxed, non-activated configuration to the expanded, activated configuration. Tube 106 may be fixed relative to proximal end 116 and movable relative to distal end 114, such that exerting a proximal force on tube 106 may cause tube 106 to transition from the non-straight configuration of FIG. 1B to the straight configuration of FIG. 1C and apply a tension to foam 104 (e.g., by pulling proximally on proximal end 116 of foam 104).

A proximal end of tube 106 may be connected to a vacuum or suction source (not shown and having any of the properties of a vacuum or suction source known in the art), which may supply a negative pressure to foam 104. For example, a negative pressure of approximately 125 mm Hg, or approximately 2.5 pounds per square inch (PSI), may be supplied to foam 104. Other suitable amounts of negative pressure may be used. The negative pressure may pull fluid, material, and/or other debris into lumen 120 of tube 106 via pores 110 of foam 104, which may promote healing of a target site. Additionally, outer wall 118 may include a plurality of openings 122 (FIGS. 1B and 1C) around a circumference of distal end 108 of tube 106 and in fluid communication with lumen 120, which may increase the flow of fluid, material, and/or other debris into lumen 120.

Additionally or alternatively, medical device 100 may include a thread 160 (e.g., a suture style thread) or other control member (e.g., a wire, cable, etc.) that is coupled to distal end 114 of foam 104. Thread 160 may extend proximally from distal end 114 of foam 104 (e.g., within foam 104), through distal opening 112 of tube 106, through lumen 120 of tube 106, and to the proximal end of tube 106. Alternatively, thread 160 may extend outside of tube 106 and alongside tube 106. A proximal end of thread 160 may be accessed by a user through, for example, the nasal or oral cavity. A distal end 162 of thread 160 may be fixed to distal end 114 of foam 104 such that proximal movement of thread 160 relative to foam 104 may provide a compressive (inward, proximal) longitudinal force on distal end 114 of foam 104 (pulling distal end 114 of foam 104 toward proximal end 116 of foam 104) and transition foam 104 to the contracted configuration.

An exemplary method of using medical device 100 will now be described. FIG. 2A illustrates an exemplary method of delivering medical device 100 to a target site 250, and FIGS. 2B and 2C illustrate medical device 100 in use within a body lumen 254 (e.g., a portion of the GI tract, e.g., the esophagus) to treat target site 250 (e.g., a wound, a leak, an esophageal fistula, etc.).

To position medical device 100 at target site 250, a user may insert an endoscope (or other medical device) into the patient via a natural orifice and position the endoscope proximate to target site 250. The user may position medical device 100 within an overtube 224 (a cross-sectional view thereof being shown in FIG. 2A) or otherwise extend medical device 100 through overtube 224. As medical device 100 is extended or advanced through a lumen 228 of overtube 224, an outer wall 226 defining lumen 228 may apply a proximal force on distal end 114 of foam 104. This force may cause foam 104 to be compressed laterally or radially inward (e.g., transition it to the contracted configuration). This contrasts to a traditional, non-auxetic foam, where such a force would cause the traditional foam to expand laterally or radially outward (bunching up the traditional foam) and making it more difficult to extend the traditional foam distally through overtube 224. The user may deliver foam 104 by pushing foam 104 distally out of a distal opening of overtube 224.

In alternative examples, the user may insert overtube 224, along with medical device 100, into a working channel of the endoscope. The user may move overtube 224, along with medical device 100, distally through the working channel. With a distal portion of the endoscope positioned proximate to target site 250, the user may move overtube 224, along with medical device 100, distally out of the working channel and position foam 104 within target site 250. Overtube 224 may then be moved proximally (retracted) to transition foam 104 from the contracted configuration to the relaxed, non-activated configuration within target site 250.

The endoscope and/or overtube 224 may then be removed from the patient's body. As shown in FIG. 2B, a size of foam 104 in the relaxed, non-activated configuration may be smaller than a size of target site 250.

The user may then connect a vacuum source (not shown) to the proximal end of tube 106 to supply a negative pressure to foam 104 through lumen 120, which may pull fluid, material, and/or other debris from target site 250 into lumen 120 to promote healing. The negative pressure supplied by tube 106 also may apply tension to foam 104 and/or result in foam 104 adhering to distal end 108 of tube 106. The user may then pull tube 106 proximally to transition distal end 108 of tube 106 from the non-linear shape (shown in FIG. 1B) to the substantially linear shape (shown in FIG. 1C). As discussed above, such a force of tube 106 may apply tension to foam 104 in one or more longitudinal directions (e.g., tube 106 may apply a proximal force on proximal end 116 of foam 104) and expand foam 104 in one or more lateral or radial directions away from the longitudinal axis of foam 104. The negative pressure supplied by tube 106 and/or the proximal movement (retraction) of tube 106 may provide the necessary tension to expand foam 104 to a size similar to a size of target site 250. The user may then leave foam 104 and tube 106 positioned within the body of the patient for a suitable amount of time.

Once treatment is completed, the user may then disconnect tube 106 from the vacuum source. Foam 104 may be transitioned to the contracted configuration via for example, pulling thread 160 coupled to distal end 114 of foam 104 proximally, as discussed above, and medical device 100 may be removed from the patient's body.

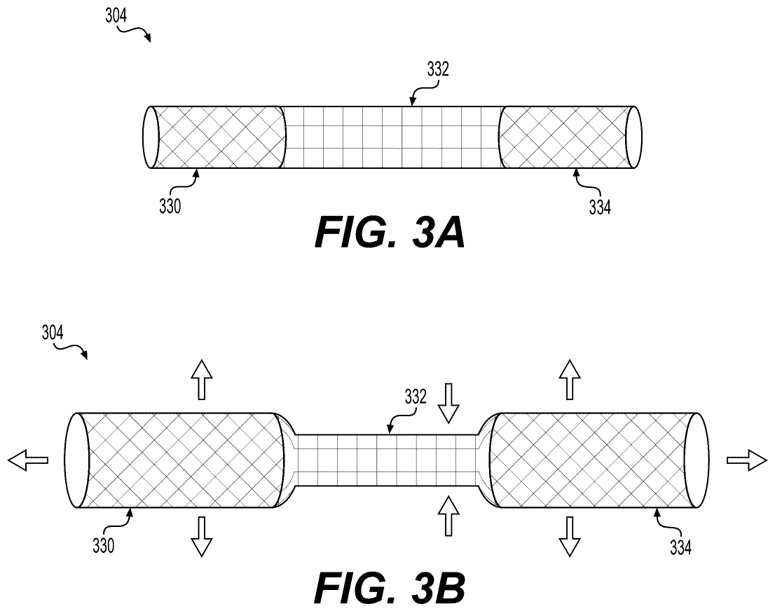

FIGS. 3A and 3B illustrate an alternative porous body or foam 304, which may have any features of foam 104, unless otherwise specified. Foam 304 may be used in conjunction with any of the aspects of medical device 100 discussed above, including for example, tube 106. Foam 304 may include sections or portions including auxetic materials and/or structures as discussed above and other sections or portions including non-auxetic materials and/or structures (e.g., any suitable material known in the art for use in EVAC that become thinner when tension is applied). Foam 304 may include a first end section or distal section 330, a second end section or proximal section 334, and a central section 332 (e.g., a saddle) positioned between end sections 330, 334. End sections 330, 334 may include auxetic materials and/or structures and central section 332 may include non-auxetic materials and/or structures. Sections 330, 332, and 334 of foam 304 may be integrally formed by a single material subject to processing such that sections 330 and 334 are converted to auxetic materials while section 332 remains of non-auxetic material. Alternatively, sections 330, 332, 334 may be formed from separate pieces of material and coupled together. It will be appreciated that the arrangement of non-auxetic and auxetic materials discussed above is merely exemplary, and any suitable portion of foam 304 may include any suitable combination of materials.

Foam 304 may be configured to transition between a relaxed, non-activated configuration (shown in FIG. 3A) and an activated (e.g., expanded) configuration (shown in FIG. 3B). Tension may be applied to foam 304 by, for example, applying a force to section 330 in a direction away from section 334 and/or applying a force to section 334 in a direction away from section 330. When foam 304 is under tension (e.g., pulled/stretched in one or more longitudinal directions shown as left and right arrows), end sections 330, 334 may expand in one or more lateral or radial directions away from a longitudinal axis of foam 304. In contrast, central section 332 may compress or contract in one or more lateral or radial directions towards the longitudinal axis of foam 304. In the relaxed, non-activated configuration, sections 330, 332, and 334 may have a same diameter. In the activated configuration, a diameter of central section 332 may be smaller than a diameter of end sections 330, 334 (e.g., foam 304 may have a dumbbell shape). The shapes described herein are merely exemplary, and foam 304 may have any suitable shape.

Foam 304 may be transitioned from the activated (e.g., expanded) configuration (FIG. 3B) to the relaxed, non-activated configuration (FIG. 3A) or to a contracted configuration (not shown) by applying a longitudinally compressive force to foam 304. For example, a force may be applied on section 330 in a direction toward section 334 and/or a force may be applied on section 334 in a direction toward section 330. Such a compressive force may cause sections 330, 334 to contract inwardly in a lateral or radial direction and may cause central section 332 to expand laterally or radially outward.

Although foam 304 is shown as having three sections 330, 332, 334, it will be appreciated that foam 304 may include any number of sections or portions having auxetic materials and/or non-auxetic materials, and having any suitable arrangement.

It will be apparent to those skilled in the art that various modifications and variations may be made in the disclosed devices and methods without departing from the scope of the disclosure. Other aspects of the disclosure will be apparent to those skilled in the art from consideration of the specification and practice of the features disclosed herein. It is intended that the specification and examples be considered as exemplary only.

We claim:

1. A medical device comprising:
a tube defining a lumen and configured to couple to a source of suction; and
a porous body coupled to a distal end of the tube, wherein the porous body includes openings in fluid communication with the lumen, and wherein, upon application of a tension force to the porous body, at least a portion of the porous body is configured to expand outward in a direction that is at least partially perpendicular to a direction of the tension force.

2. The medical device of claim 1, wherein the porous body includes an auxetic material.

3. The medical device of claim 2, wherein the openings of the porous body have a re-entrant structure.

4. The medical device of claim 1, wherein the tension force is at least one of a proximal force on a proximal end of the porous body or a distal force on a distal end of the porous body.

5. The medical device of claim 1, wherein the distal end of the tube is configured to transition from a non-linear shape to a linear shape in order to apply the tension force on the porous body.

6. The medical device of claim 1, wherein the openings of the porous body are configured to expand in a direction perpendicular to the tension force upon application of the tension force to the porous body.

7. The medical device of claim 1, wherein, upon application of a compressive force, the at least the portion of the porous body is configured to contract inwardly in a direction that is at least partially perpendicular to the compressive force, and wherein the compressive force is in an opposite direction from the direction of the tension force.

8. The medical device of claim 1, further comprising a suture thread extending proximally from a distal end of the porous body, through the lumen of the tube, and to a proximal end of the tube.

9. The medical device of claim 8, wherein a distal end of the suture thread is fixedly coupled to the distal end of the porous body.

10. The medical device of claim 9, wherein, upon movement of the suture thread in a proximal direction, the at least the portion of the porous body is configured to contract inwardly in a direction that is at least partially perpendicular to the proximal direction.

11. The medical device of claim 1, wherein the porous body includes a first section and a second section, wherein the first section includes an auxetic material, wherein the second section includes a non-auxetic material, and wherein the first section includes the at least the portion of the porous body that is configured to expand outward in the direction that is at least partially perpendicular to the direction of the tension force.

12. The medical device of claim 11, wherein the porous body further includes a third section, wherein the second section is positioned between the first section and the third section, and wherein the third section includes an auxetic material.

13. The medical device of claim 12, wherein, upon application of the tension force, the third section is configured to expand outward in the direction that is at least partially perpendicular to the direction of the tension force, and the second section is configured to contract inwardly in a direction that is at least partially perpendicular to the direction of the tension force.

14. The medical device of claim 13, wherein, after application of the tension force, the third section and the first section have a larger diameter than the second section.

15. The medical device of claim 12, wherein, prior to application of the tension force, the first section, the second section, and the third section have approximately a same diameter.

16. A medical device comprising:
a tube defining a lumen and configured to couple to a source of suction; and
a porous body including one or more materials and coupled to a distal end of the tube, wherein the porous body includes openings in fluid communication with the lumen, and wherein the porous body includes an auxetic material.

17. The medical device of claim 16, wherein, upon application of a compressive force to the porous body, at least a portion of the porous body including the auxetic material is configured to contract radially inwardly in a direction that is at least partially perpendicular to the compressive force.

18. A method of treating a target site of a body lumen of a patient via a medical device, the method comprising:
advancing a porous body to the target site, wherein the porous body is coupled to a distal end of tube;
supplying a negative pressure to the porous body through a lumen of the tube; and
pulling the tube in a proximal direction such that at least a portion of the porous body expands outwardly in a direction that is at least partially perpendicular to the proximal direction.

19. The method of claim 18, wherein pulling the tube in the proximal direction transitions the tube from a non-linear to a linear shape.

20. The method of claim 18, wherein the direction is a first direction, the method further comprising pulling a suture coupled to a distal end of the porous body in the proximal direction, thereby causing the at least the porous body to contract inwardly in a second direction that is at least partially perpendicular to the proximal direction.
